(19) United States
(12) Patent Application Publication
(10) Pub. No.: US 2014/0107327 A1
(43) Pub. Date: Apr. 17, 2014

(54) METHODS FOR PREPARING DEUTERATED 1,2,3-TRIAZOLES

(71) Applicants: Mahesh K. Lakshman, Teaneck, NJ (US); Hari Kiran Akula, Astoria, NY (US)

(72) Inventors: Mahesh K. Lakshman, Teaneck, NJ (US); Hari Kiran Akula, Astoria, NY (US)

(73) Assignee: RESEARCH FOUDATION OF THE CITY UNIVERSITY OF NEW YORK, New York, NY (US)

(21) Appl. No.: 14/007,537

(22) PCT Filed: Mar. 26, 2012
(86) PCT No.: PCT/US12/30551

§ 371 Date: Dec. 16, 2013

Related U.S. Application Data
(60) Provisional application No. 61/468,190, filed on Mar. 28, 2011.

(51) Int. Cl. C07H 19/16; C07D 403/06; C07D 249/04; C07F 7/02; C07D 401/04

(52) U.S. Cl. CPC C07H 19/16; C07F 7/02; C07D 401/04; C07D 249/04; C07D 403/06
USPC 536/17.4; 544/229; 546/268.4; 548/255

(57) ABSTRACT

This disclosure relates to a method that involves reacting an azide with an alkyne in the presence of deuterated water and a copper-containing catalyst, thereby forming a deuterated 1,2,3-triazole.

### CROSS REFERENCE TO RELATED APPLICATION

Pursuant to 35 U.S.C. §119(e), this application claims priority to U.S. Provisional Application Ser. No. 61/468,190, filed Mar. 28, 2011, the entire contents of which are hereby incorporated by reference.

### STATEMENT REGARDING FEDERALLY SPONSORED RESEARCH

This invention was supported by Grant No. 1R21 AI094545-01 awarded by National Institute of Health. The U.S. Government has certain rights in the invention.

### TECHNICAL FIELD

This disclosure relates to deuterated 1,2,3-triazoles and methods for preparing these compounds.

### BACKGROUND

1,2,3-triazolyl moiety is an important motif in pharmaceuticals. For example, Tazobactam and Cefatrizine are β-lactamase inhibitors, Rufinamide is an anti-epileptic agent, and carboxyamidotriazole is an anticancer agent that decreases VEGF expression. Potent new HIV protease inhibitors, inhibitors of carbonic anhydrase and acetylcholine esterase, ceramide analogues, tubulin disrupting podophyllotoxin analogues, selective anti-HCV agents, and many other therapeutically valuable entities containing the 1,2,3-triazolyl moiety have been discovered.

More recently, incorporation of deuterium (D) has gained visibility in medicinal chemistry. “Heavy drugs” are pharmaceutical entities with one or more H atoms replaced deuterium. One underlying reason for the emergence of these isotopically heavy compounds is the higher C-D bond energy compared the C—H bond energy, which makes the C-D bond cleavage more difficult.

### SUMMARY

This disclosure is based on an unexpected discovery that certain deuterated 1,2,3-triazoles can be prepared by a facile and regioselective reaction between a suitable azide and a suitable alkyne in the presence of deuterated water and a copper-containing catalyst. The deuterated 1,2,3-triazoles can have therapeutic effects and be used in pharmaceuticals.

In one aspect, the disclosure features a method that includes reacting an azide with an alkyne in the presence of deuterated water and a copper-containing catalyst, thereby forming a deuterated 1,2,3-triazole.

In another aspect, the disclosure features a method that includes preparing a deuterated 1,2,3-triazole of formula (I):

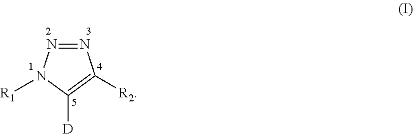

The method includes reacting an azide of formula (II): R₁—N₃ (II) with an alkyne of formula (III):

R₃≡R₂ (III)

in the presence of deuterated water and a copper-containing catalyst, thereby forming the deuterated 1,2,3-triazole of formula (I), in which R₁ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group; R₂ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof; and R₃ is H or D.

In still another aspect, the disclosure features a compound of formula (I):

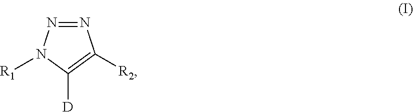

in which R₁ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group, provided that R₁ is not C₁-C₁₀ alkyl substituted with phenyl; and R₂ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof.

Embodiments can include one or more of the following features.

The deuterated 1,2,3-triazole can contain a deuterium atom at the 5 position.

The alkyne can be ethyne substituted with a substituent comprising C₁-C₂₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₂₀ alkylstannyl, or a boronic acid group or an ester thereof. In some embodiments, the substituent can include C₁-C₁₀ alkyl, C₁-C₂₀ heterocycloalkyl, aryl, or heteroaryl, which is optionally substituted with D, halo (e.g., F, Cl, Br, or I), CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, the substituent can include pyridyl, CH₃ substituted with

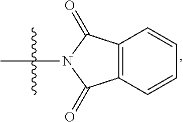 [SMILES: *N1C(=O)c2ccccc2C1=O]

or phenyl optionally substituted with D, F, or CH₃.

The azide can include C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group. In some embodiments, the azide can include aryl, a nucleoside group, or a deoxynucleoside group, which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₂₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, the azide can include naphthyl, an adenosine group optionally substituted with C₁-C₁₀ alkylsilyl (e.g., tert-butyldimethylsilyl), C₁-C₁₀ alkyl, or C₁-C₁₀ acyl, a thymidine group optionally substituted with C₁-C₁₀ alkylsilyl (e.g., tert-butyldimethylsilyl), C₁-C₁₀ alkyl, or C₁-C₁₀ acyl, or phenyl optionally substituted with D, CN, C₁-C₁₀ alkyl (e.g., tert-butyl), or C₁-C₁₀ alkoxy (e.g., methoxy).

The catalyst can include a copper (I) ion or a copper (II) ion. In some embodiments, when the catalyst includes a copper (II) ion, the reacting step is conducted in the presence of a reducing agent (e.g., sodium ascorbate). In some embodiments, the catalyst is at least about 5 mol % of the azide.

The reacting step is conducted in the presence of an aprotic solvent. In some embodiments, the aprotic solvent is immiscible with water. Exemplary aprotic solvents include methylene chloride, 1,2-dichloroethane, ethyl acetate, chloroform, benzene, an alkyl benzene, or a halo benzene.

R₁ can be aryl, a nucleoside group, or a deoxynucleoside group, which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, R₁ can be naphthyl, an adenosine group optionally substituted with C₁-C₁₀ alkylsilyl (e.g., tert-butyldimethylsilyl), C₁-C₁₀ alkyl, or C₁-C₁₀ acyl, a thymidine group optionally substituted with C₁-C₁₀ alkylsilyl (e.g., tert-butyldimethylsilyl), C₁-C₁₀ alkyl, or C₁-C₁₀ acyl, or phenyl optionally substituted with D, CN, C₁-C₁₀ alkyl (e.g., tert-butyl), or C₁-C₁₀ alkoxy (e.g., methoxy).

R₂ can be C₁-C₁₀ alkyl, C₁-C₂₀ heterocycloalkyl, aryl, or heteroaryl, which is optionally substituted with D, halo, CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, R₂ can be pyridyl, CH₃ substituted with

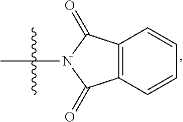 [SMILES: *N1C(=O)c2ccccc2C1=O]

or phenyl optionally substituted with D, F, or CH₃.

Other features, objects, and advantages of the subject matter in this disclosure will be apparent from the description and the claims.

### DETAILED DESCRIPTION

In general, this disclosure relates to facile and regioselective methods of preparing deuterated 1,2,3-triazoles by reacting an azide and an alkyne in the presence of deuterated water and a copper-containing catalyst. In some embodiments, the deuterated 1,2,3-triazoles can contain a deuterium atom at the 5-position. As used herein, the term “5-position” refers to the corresponding position shown in Formula (I) above.

The alkyne suitable for use in the methods disclosed herein can be ethyne substituted with a substituent containing C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof. For example, the substituent can be C₁-C₁₀ alkyl (e.g., CH₃ substituted with

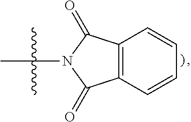 [SMILES: *N1C(=O)c2ccccc2C1=O]

C₁-C₂₀ heterocycloalkyl, aryl (e.g., phenyl optionally substituted with D, F, or CH₃), or heteroaryl (e.g., pyridyl), which can be optionally further substituted with D, halo, CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

The term “alkyl” refers to a saturated, linear or branched hydrocarbon moiety, such as —CH₃ or —CH(CH₃)₂. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), naphthyl, pyrenyl, anthryl, and phenanthryl. The term “heteroaryl” refers to a moiety having one or more aromatic rings that contain at least one heteroatom (e.g., N, O, or S). Examples of heteroaryl moieties include furyl, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl also include those fused with one or more additional rings.

In some embodiments, the alkyne suitable for use in the methods disclosed herein can be ethyne that is substituted with one of the substituents described at one end and is unsubstituted or substituted with D at the other end (i.e., containing a —C≡CH or —C≡CD group). Without wishing to be bound by theory, it is believed that, when an alkyne containing a —C≡CH group is used in the methods disclosed herein, the proton in —C≡CH can generate water (H₂O or HOD) with deuterated water during the reaction between the alkyne and the azide. In a large scale manufacturing process, the water thus generated could subsequently react with a cuprated 1,2,3-triazole intermediate (i.e., a 1,2,3-triazole containing a copper ion attached at 5-position of the triazole ring) to form a 1,2,3-triazole containing proton (instead of deuterium) at the 5-position, thereby reducing the extent of deuterium incorporation and/or the yield of the deuterated 1,2,3-triazole. Thus, without wishing to be bound by theory, it is believed that using an alkyne containing —C≡CD could avoid producing H₂O or HOD generated from the proton in —C≡CH and therefore minimize the side reaction described above.

In some embodiments, when the alkyne is substituted with C₁-C₁₀ alkylsilyl, C₁-C₁₀ alkylstannyl, or a boronic acid group or an ester thereof, the deuterated 1,2,3-triazole formed by the methods disclosed herein can further react with a suitable reagent to form another deuterated 1,2,3-triazole. For example, when the alkyne is substituted with C₁-C₁₀ alkylsilyl (e.g., t-butyldimethylsilyl or trimethylsilyl), the deuterated 1,2,3-triazole formed by the methods disclosed herein can further react with a suitable reagent (e.g., HF) to convert the alkylsilyl group to a proton. Alternatively, the silyl group can be replaced with a hydrocarbon via metal-catalyzed processes such as a Hiyama cross-coupling reaction. As another example, when the alkyne is substituted with C₁-C₁₀ alkylstannyl (e.g., tert-butyldimethylstannyl or tri-n-butylstannyl), the deuterated 1,2,3-triazole formed by the methods disclosed herein can further react with a suitable reagent (e.g., a halogenated (Cl, Br, or I containing) hydrocarbon) to convert the alkylstannyl group to a hydrocarbon moiety via processes such as metal catalyzed Stille reactions.

The azide suitable for use in the methods disclosed herein can include C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group. A subset of the suitable azide can be those including aryl (e.g., naphthyl or phenyl), a nucleoside group (e.g., adenosine), or a deoxynucleoside group (e.g., thymidine), which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, the azide can include naphthyl, an adenosine group optionally substituted with C₁-C₁₀ alkylsilyl (e.g., one or more tert-butyldimethylsilyl groups), C₁-C₁₀ alkyl, or C₁-C₁₀ acyl, a thymidine group optionally substituted with C₁-C₁₀ alkylsilyl (e.g., one or more tert-butyldimethylsilyl groups), C₁-C₁₀ alkyl, or C₁-C₁₀ acyl, or phenyl optionally substituted with D, CN, C₁-C₁₀ alkyl (e.g., tert-butyl), or C₁-C₁₀ alkoxy (e.g., methoxy).

The copper-containing catalyst suitable for use in the methods disclosed herein can include a copper (I) ion or a copper (II) ion. Exemplary suitable copper-containing catalysts include copper (I) salts and copper (II) salts. An example of a suitable catalyst containing a copper (II) ion is copper sulfate (e.g., anhydrous copper sulfate). An example of a suitable catalyst containing a copper (I) ion is copper monohalide (e.g., anhydrous copper monochloride). Without wishing to be bound by theory, it is believed that using an anhydrous copper-containing catalyst can reduce the amount of residual water in the methods disclosed herein (which would interfere the deuteration reaction between D₂O and a cuprated 1,2,3-triazole intermediate formed by the azide and alkyne) and therefore improve deuterium incorporation and/or the yield of the deuterated 1,2,3-triazole.

In some embodiments, the catalyst can include a complex formed between copper (I) and suitable ligands. Exemplary suitable ligands include halo (e.g., Cl, Br, or I), PPh₃,

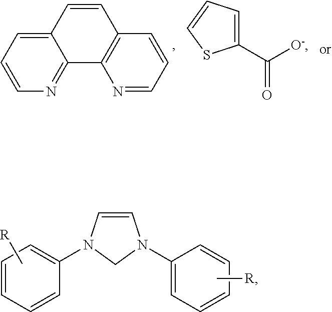

in which R is C₁-C₁₀ alkyl. Examples of such complexes include:

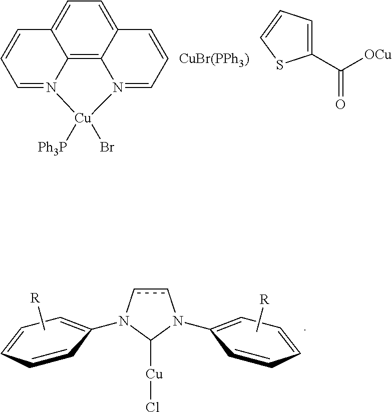

In some embodiments, when the catalyst includes a copper (II) ion, the methods disclosed herein can be performed in the presence of a reducing agent. An example of such a reducing agent is sodium ascorbate or copper (Cu). Without wishing to be bound by theory, it is believed that the reducing agent can reduce the copper (II) ion to a copper (I) ion, which is like to be an important species that catalyzes the reaction between the alkyne and the azide.

In general, the amount of the copper-containing catalyst is large enough to substantially complete the reaction between the alkyne and the azide used in the methods disclosed herein. For example, the catalyst can be at least about 5 mol % (e.g., at least about 7 mol %, at least about 10 mol %, or at least about 20 mol %) of the amount of the azide used in the methods disclosed herein. In some embodiment, when the reducing agent is present in the methods disclosed herein, the amount of the reducing agent can be at least about 5 mol % (e.g., at least about 10 mol %, at least about 15 mol %, or at least about 20 mol %) of the amount of the azide.

In some embodiments, the methods disclosed herein can be performed in the presence of an aprotic solvent. Examples of suitable aprotic solvents include methylene chloride, 1,2-dichloroethane, ethyl acetate, chloroform, benzene, an alkyl benzene (e.g., toluene or xylene), or a halo benzene (e.g. chlorobenzene or dichlorobenzene). In some embodiments, the aprotic solvent is immiscible with water. As used herein, “a solvent immiscible with water” refers to a solvent that forms two phases when mixed with water. In such embodiments, the methods disclosed herein can be carried out in a two-phase reaction system, in which the azide and the alkyne are dissolved in the aprotic solvent, while the copper-containing catalyst (e.g., anhydrous copper sulfate) and the optional reducing agent (e.g., sodium ascorbate) are dissolved in deuterated water. Without wishing to be bound by theory, it is believed that using a two-phase reaction system can significantly reduce side reactions, enhance the reaction rate, and improve the extent of deuterium incorporation and/or the yield of the deuterated 1,2,3-triazole. In some embodiments, an aprotic solvent miscible with water can be used in the methods disclosed herein.

Without wishing to be bound by theory, one advantage of the methods disclosed herein is that the methods can be performed by using inexpensive reagents (e.g., CuSO₄, sodium ascorbate, CH₂Cl₂, and D₂O) that do not require special handling other than minimizing contact with moisture. By contrast, conventional methods of preparing deuterated 1,2,3-triazoles generally require use of flammable or pyrophoric organometallic reagents, which require special handling. In addition, without wishing to be bound by theory, another advantage of the methods disclosed herein is that the methods can be readily performed at room temperature in one step. By contrast, conventional methods of preparing deuterated 1,2,3-triazoles are generally performed in multiple steps at temperatures higher or lower than room temperature, which would increase costs during large scale manufacturing. In addition, the methods disclosed herein can also be used to prepare deuterated 1,2,3-triazoles containing moieties (e.g., nucleosides) that are easily degraded under harsh reaction conditions. By contrast, conventional methods of preparing deuterated 1,2,3-triazoles generally uses harsh reaction conditions and therefore may not be used for preparing such compounds.

Scheme 1 below depicts an exemplary method of preparing deuterated 1,2,3-triazoles.

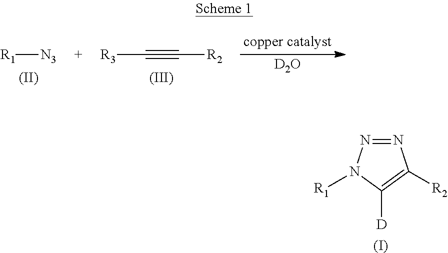

As shown in scheme 1, the method is performed by reacting an azide of formula (II) with an alkyne of formula (III) in the presence of deuterated water and a copper-containing catalyst, thereby forming the deuterated 1,2,3-triazole of formula (I). In scheme 1, R₁ can be C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group; R₂ can be C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof; and R₃ can be H or D.

In a subset of the azides of formula (II), R₁ can be aryl, a nucleoside group, or a deoxynucleoside group, which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, R₁ can be naphthyl, an adenosine group optionally substituted with C₁-C₁₀ alkylsilyl (e.g., tert-butyldimethylsilyl), C₁-C₁₀ alkyl, or C₁-C₁₀ acyl, a thymidine group optionally substituted with C₁-C₁₀ alkylsilyl (e.g., tert-butyldimethylsilyl), C₁-C₁₀ alkyl, or C₁-C₁₀ acyl, or phenyl optionally substituted with D, CN, C₁-C₁₀ alkyl (e.g., tert-butyl), or C₁-C₁₀ alkoxy (e.g., methoxy).

In a subset of the alkynes of formula (III), R₂ can be C₁-C₁₀ alkyl, C₁-C₂₀ heterocycloalkyl, aryl, or heteroaryl, which is optionally substituted with D, halo, CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. For example, R₂ can be pyridyl, CH₃ substituted with

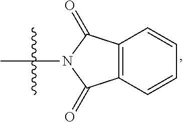 [SMILES: *N1C(=O)c2ccccc2C1=O]

or phenyl optionally substituted with D, F, or CH₃.

Also within the scope of this disclosure are the deuterated 1,2,3-triazoles of formula (I), in which R₁ and R₂ are described above provided that R₁ is not C₁-C₂₀ alkyl substituted with phenyl. These compounds can have therapeutic effects and therefore can be used as pharmaceuticals, such as anti-cancer agents or anti-epileptic agents. Without wishing to be bound by theory, it is believed that the deuterated 1,2,3-triazoles of formula (I) can have improved pharmacokinetics and therefore improved potency compared to their un-deuterated analogs due to the presence of C-D bond.

Shown below are exemplary compounds 1-14 of deuterated 1,2,3-triazoles of formula (I). Details of preparation of these compounds are provided in Examples 1-14 below, respectively.

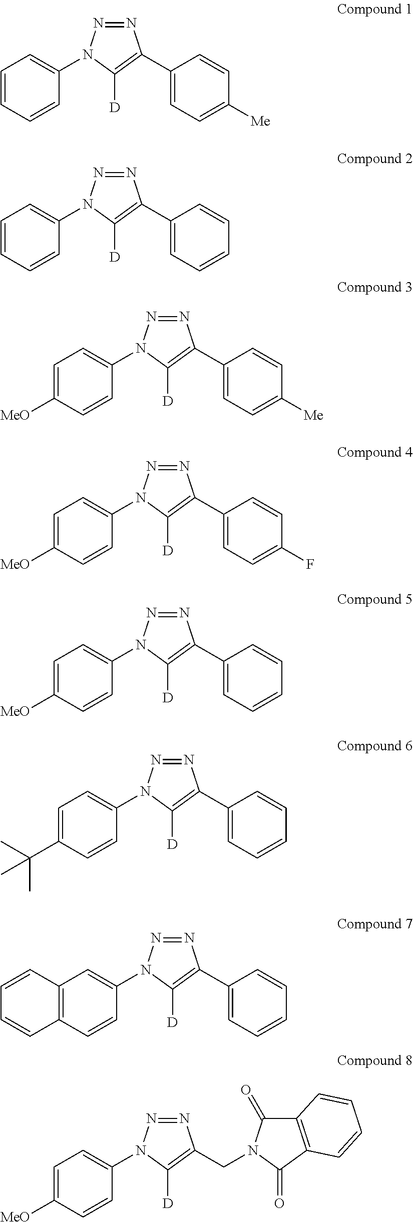

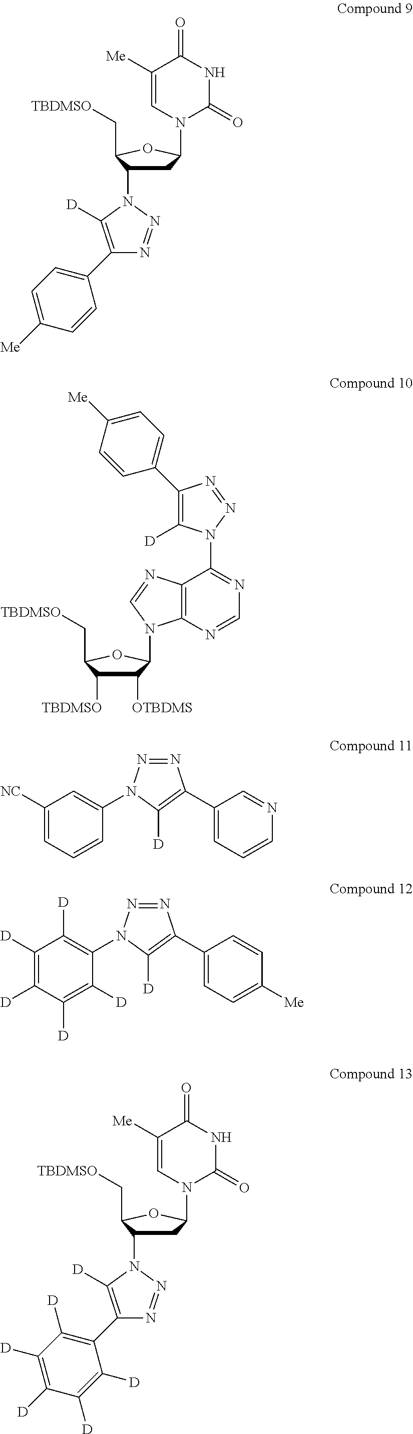

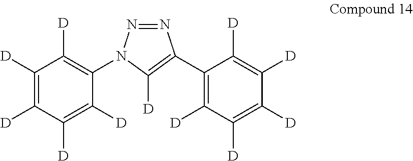 [SMILES: [2H]c1c([2H])c([2H])c(-c2nnn(-c3c([2H])c([2H])c([2H])c([2H])c3[2H])c2[2H])c([2H])c1[2H]]

A deuterated 1,2,3-triazole synthesized by the methods described herein can be purified by a suitable method such as column chromatography, high-pressure liquid chromatography, or recrystallization.

Other deuterated 1,2,3-triazoles can be prepared using suitable starting materials through the methods described herein. The methods described herein may additionally include steps, either before or after the steps described specifically herein, to add or remove suitable protecting groups, or to introduce additional substituent groups in order to ultimately allow synthesis of the deuterated 1,2,3-triazoles. Synthetic chemistry transformations and protecting group methodologies (protection and deprotection) useful in synthesizing applicable deuterated 1,2,3-triazoles are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The deuterated 1,2,3-triazoles mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this disclosure is a pharmaceutical composition containing an effective amount of at least one deuterated 1,2,3-triazole described herein and a pharmaceutical acceptable carrier. Further, this disclosure includes a method of administering an effective amount of one or more of the deuterated 1,2,3-triazoles to a patient having a disease (e.g., an inherited or acquired disease). Examples of diseases that can be treated by the deuterated 1,2,3-triazoles include cancer (e.g., breast, lung, colon, stomach, or ovarian cancer, or leukemia), epilepsy, or a viral disease. “An effective amount” refers to the amount of an active deuterated 1,2,3-triazole that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the pharmaceutical composition described, a composition having one or more deuterated 1,2,3-triazoles can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active deuterated 1,2,3-triazoles can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active deuterated 1,2,3-triazole. Examples of other carriers include colloidal silicon oxide, magnesium stearate, cellulose, sodium lauryl sulfate, and D&C Yellow #10.

The deuterated 1,2,3-triazoles described herein can be preliminarily screened for their efficacy in treating an above-described disease by an in vitro assay and then confirmed by animal experiments and clinic trials. Other methods will also be apparent to those of ordinary skill in the art.

The methods and the deuterated 1,2,3-triazoles described herein can also be used in isotopic labeling in mechanistic studies, metabolic studies, and mass spectrometric applications.

The contents of all publications cited herein (e.g., patents, patent application publications, and articles) are hereby incorporated by reference in their entirety.

The following examples are illustrative and not intended to be limiting.

### EXAMPLES

### General Experimental Considerations

Thin layer chromatography (TLC) was performed on aluminum foil-backed TLC plates of 200 μm thickness and column chromatographic purifications were performed on 200-300 mesh silica gel. Prior to use, CH₂Cl₂ and MeCN were distilled over CaH₂, Et₂O was distilled over LiAlH₄ and then over Na. All other reagents were obtained from commercial sources and were used without further purification. ¹H NMR spectra were recorded at 500 MHz and are referenced to the residual protonated solvent resonance. ¹³C NMR spectra were recorded at 125 MHz in CDCl₃ and are referenced to the solvent resonance. ²H NMR spectra were recorded at 77 MHz using CDCl₃ as an internal standard. In particular, the ¹H NMR spectrum of CDCl₃ was first recorded at 500 MHz and the residual protonated solvent resonance was set to δ 7.26 ppm. The ²H NMR of the same sample was then recorded at 77 MHz, and this showed a resonance at δ 7.24 ppm. From this analysis, CDCl₃ was referred to δ 7.24 ppm for all ²H NMR experiments. ¹⁹F NMR spectra were recorded at 282 MHz using CFCl₃ as internal standard. Chemical shifts (6) are reported in parts per million (ppm) and coupling constants (J) are in hertz (Hz). The alkynes and azides used in the examples were either purchased from a commercial source or synthesized by the methods described herein.

### General Procedure for Synthesizing Deuterated 1,2,3-Triazoles

An appropriate azide (0.335 mmol) in 1.6 mL of dry CH₂Cl₂ was placed a clean, extremely dry, 10 mL round-bottom flask equipped with a stirring bar. To this stirred solution, an appropriate alkyne (0.67 mmol) was added. In a separate vial, CuSO₄.5H₂O (8.4 mg, 0.0335 mmol, 10 mol % of the amount of the azide) was heated at 120° C. under vacuum until its color changed from blue to grey. Using this dehydrated CuSO₄, a 0.048 M solution was prepared in a glove bag by the addition of 0.7 mL 99.8% D₂°, and this solution was transferred to the above reaction mixture. After a 0.098 M solution of Na ascorbate (13.3 mg in 0.7 mL of 99.8% D₂O, 20 mol % of the amount of the azide) was added, the reaction mixture was sealed and stirred under nitrogen gas at room temperature until TLC showed consumption of the azide. The organic layer of the reaction mixture was separated and the aqueous layer was extracted with ethyl acetate (3×5 mL). The combined organic layers were washed with brine (5 mL), dried over anhydrous Na₂SO₄, and concentrated under reduced pressure. The crude material was purified by chromatography on a silica gel column.

### Example 1

### Preparation of Compound 1: 5-Deutero-1-phenyl-4-(p-tolyl)-1H-1,2,3-triazole

Compound 1 was prepared by using phenyl azide and p-tolyl ethyne following the general synthetic procedure described above. Phenyl azide was synthesized from its corresponding boronic acid using the procedures described in Tao et al., Tetrahedron Lett. 2007, 48, 3525-3529. The final product was purified by column chromatography using hexane followed by 20% EtOAc in hexane to give an off-white solid (Yield: 75%; % D: 95%). R_{f} (SiO₂/25% EtOAc in hexanes)=0.34. ¹H NMR (500 MHz, CDCl₃): δ 7.82-7.79 (m, 4H, Ar—H), 7.55 (t, 2H, Ar—H, J=7.4 Hz), 7.46 (t, 1H, Ar—H, J=7.3 Hz), 7.28 (d, 2H, Ar—H, J=7.4 Hz), 2.41 (s, 3H, CH₃). ¹³C NMR (125 MHz, CDCl₃): δ 148.7, 138.5, 137.5, 129.9, 129.8, 128.8, 127.8, 126.1, 120.7, 117.2 (t, J=30.4 Hz), 21.4. ²H NMR (77 MHz, CHCl₃): δ 8.19. HRMS calcd for C₁₅H₁₃DN₃ [M+H]⁺ 237.1245. found 237.1248.

### Example 2

### Preparation of Compound 2: 5-Deutero-1,4-diphenyl-1H-1,2,3-triazole

Compound 2 was prepared by using phenyl azide and phenyl ethyne following the general synthetic procedure described above. Phenyl azide was synthesized from its corresponding boronic acid using the procedures described in Tao et al., Tetrahedron Lett. 2007, 48, 3525-3529. The final product was purified by column chromatography using hexane followed by 20% EtOAc in hexane to give an off-white solid (Yield: 72%; % D: 94%). R_{f} (SiO₂/25% EtOAc in hexanes)=0.34. ¹H NMR (500 MHz, CDCl₃): δ 7.92 (d, 2H, Ar—H, J=8.1 Hz), 7.80 (d, 2H, Ar—H, J=7.8 Hz), 7.55 (t, 2H, Ar—H, J=7.8 Hz), 7.48-7.45 (m, 3H, Ar—H), 7.37 (t, 1H, Ar—H, J=7.4 Hz). ¹³C NMR (125 MHz, CDCl₃): δ 148.6, 137.4, 130.6, 130.0, 129.1, 128.9, 128.6, 126.2, 120.8, 117.6 (s, J=28.1 Hz). ²H NMR (77 MHz, CHCl₃): δ 8.22. HRMS calcd for C₁₄H₁₁DN₃ [M+H]⁺ 223.1089. found 223.1086.

### Example 3

### Preparation of Compound 3: 5-Deutero-1-(4-methoxyphenyl)-4-(p-tolyl)-1H-1,2,3-triazole

Compound 3 was prepared by using 4-methoxyphenyl azide and p-tolyl ethyne following the general synthetic procedure described above. 4-Methoxyphenyl azide was synthesized from its corresponding boronic acid using the procedures described in Tao et al., Tetrahedron Leu. 2007, 48, 3525-3529. The final product was purified by column chromatography using hexane followed by 20% EtOAc in hexane to give an off-white solid (Yield: 87%; % D: 97%). R_{f} (SiO₂/25% EtOAc in hexanes)=0.26. ¹H NMR (500 MHz, CDCl₃): δ 7.80 (d, 2H, Ar—H, J=7.6 Hz), 7.67 (d, 2H, Ar—H, J=8.6 Hz), 7.26 (d, 2H, Ar—H, J=7.6 Hz), 7.02 (d, 2H, Ar—H, J=8.6 Hz), 3.87 (s, 3H, OCH₃), 2.40 (s, 3H, CH₃). ¹³C NMR (125 MHz, CDCl₃): δ 159.9, 148.3, 138.4, 130.7, 129.7, 127.7, 125.9, 122.3, 117.5 (t, J=28.9 Hz), 114.9, 55.8, 21.5. ²H NMR (77 MHz, CHCl₃): δ 8.10. HRMS calcd for C₁₆H₁₅DN₃O [M+H]⁺ 267.1351. found 267.1356.

### Example 4

### Preparation of Compound 4: 5-Deutero-4-(4-fluorophenyl)-1-(4-methoxyphenyl)-1H-1,2,3-triazole

Compound 4 was prepared by using 4-methoxyphenyl azide and 4-fluorophenyl ethyne following the general synthetic procedure described above. 4-Methoxyphenyl azide was synthesized from its corresponding boronic acid using the procedures described in Tao et al., Tetrahedron Lett. 2007, 48, 3525-3529. The final product was purified by column chromatography using hexane followed by 20% EtOAc in hexane to give an off-white solid (Yield: 70%; % D: 96%). R_{f} (SiO₂/25% EtOAc in hexanes)=0.22. ¹H NMR (500 MHz, CDCl₃): δ 7.89-7.86 (m, 2H, Ar—H), 7.67 (d, 2H, Ar—H, J=8.9 Hz), 7.14 (t, 2H, Ar—H, J=8.6 Hz), 7.04 (d, 2H, Ar—H, J=8.9 Hz), 3.88 (s, 3H, OCH₃). ¹³C NMR (125 MHz, CDCl₃): δ 164.1, 162.1, 160.3, 147.5, 130.8, 127.8 (d, J=8.1 Hz), 127.0 (d, J=3.2 Hz), 122.4, 117.6 (t, J=28.9 Hz), 116.1 (d, J=21.8 Hz), 115.1, 55.9. ²H NMR (77 MHz, CHCl₃): δ 8.09. ¹⁹F NMR (282 MHz, CDCl₃): δ−113.8 (relative to CFCl₃). HRMS calcd for C₁₅H₁₂DFN₃O [M+H]⁺ 271.1100. found 271.1105.

### Example 5

### Preparation of Compound 5: 5-Deutero-1-(4-methoxyphenyl)-4-phenyl-1H-1,2,3-triazole

Compound 5 was prepared by using 4-methoxyphenyl azide and phenyl ethyne following the general synthetic procedure described above. 4-Methoxyphenyl azide was synthesized from its corresponding boronic acid using the procedures described in Tao et al., Tetrahedron Lett. 2007, 48, 3525-3529. The final product was purified by column chromatography using hexane followed by 20% EtOAc in hexane to give an off-white solid (Yield: 91%; % D: 97%). R_{f} (SiO₂/25% EtOAc in hexanes)=0.23. ¹H NMR (500 MHz, CDCl₃): δ 7.90 (d, 2H, Ar—H, J=7.8 Hz), 7.69 (d, 2H, Ar—H, J=8.9 Hz), 7.46 (t, 2H, Ar—H, J=7.6 Hz), 7.37 (t, 1H, Ar—H, J=7.4 Hz), 7.04 (d, 2H, Ar—H, J=8.9 Hz), 3.88 (s, 3H, OCH₃). ¹³C NMR (125 MHz, CDCl₃): δ 160.2, 148.4, 130.9, 130.8, 129.1, 128.5, 126.1, 122.4, 117.8 (t, J=28.3 Hz), 115.1, 55.9. ²H NMR (77 MHz, CHCl₃): δ 8.16. HRMS calcd for C₁₅H₁₃DN₃O [M+H]⁺ 253.1194. found 253.1199.

### Example 6

### Preparation of Compound 6: 5-Deutero-1-(4-(tert-butyl)phenyl)-4-phenyl-1H-1,2,3-triazole

Compound 5 was prepared by using 4-(tert-butyl)phenyl azide and phenyl ethyne following the general synthetic procedure described above.

4-(Tert-butyl)phenyl azide was prepared as follows: In a clean 10 mL round-bottom flask equipped with a stirring bar, 4-(tert-butyl)phenylboronic acid (356 mg, 2 mmol) was dissolved in MeOH (6 mL). After NaN₃ (156 mg, 2.4 mmol) and CuSO₄.5H₂O (50 mg, 0.2 mmol) were added to the reaction mixture, the mixture was stirred in an open flask at room temperature for 16 hours. The solvent was evaporated under reduced pressure and EtOAc (10 mL) was added. The mixture was then extracted with H₂O (10 mL) and the aqueous layer was extracted with EtOAc (3×10 mL). The combined organic layers were dried over anhydrous Na₂SO₄ and concentrated under reduced pressure. The crude product was purified by column chromatography on a short silica gel plug using hexane as an eluent to give the 4-(tert-butyl)phenyl azide as an orange-red liquid (Yield: 311.5 mg; 89%). R_{f} (SiO₂/hexanes)=0.6×. ¹H NMR (500 MHz, CDCl₃): δ 7.38 (d, 2H, Ar—H, J=8.6 Hz), 6.98 (d, 2H, Ar—H, J=8.6 Hz), 1.32 (s, 9H, tert-Bu).

The final product was purified by column chromatography using hexane followed by 20% EtOAc in hexane to give an off-white solid (Yield: 85%; % D: 96%). R_{f} (SiO₂/25% EtOAc in hexanes)=0.46. ¹H NMR (500 MHz, CDCl₃): δ 7.92 (d, 2H, Ar—H, J=8.1 Hz), 7.70 (d, 2H, Ar—H, J=8.5 Hz), 7.53 (d, 2H, Ar—H, J=8.5 Hz), 7.44 (t, 2H, Ar—H, J=7.6 Hz), 7.35 (dt, 1H, Ar—H, J=1.0, 7.4 Hz), 1.38 (s, 9H, tert-Bu). ¹³C NMR (125 MHz, CDCl₃): δ 152.3, 148.3, 134.8, 130.5, 129.1, 128.5, 126.8, 126.0, 120.4, 117.8 (t, J=28.9 Hz), 35.0, 31.5. ²H NMR (77 MHz, CHCl₃): δ 8.20. HRMS calcd for C₁₈H₁₉DN₃ [M+H]⁺ 279.1715. found 279.1709.

### Example 7

### Preparation of Compound 7: 5-Deutero-1-(naphthalen-1-yl)-4-phenyl-1H-1,2,3-triazole

Compound 7 was prepared by using 1-naphthyl azide and phenyl ethyne following the general synthetic procedure described above. 1-Naphthyl azide was synthesized from its corresponding boronic acid using the procedures described in Tao et al., Tetrahedron Lett. 2007, 48, 3525-3529. The final product was purified by column chromatography using hexane followed by 20% EtOAc in hexane to give an off-white solid (Yield: 72%; % D: 92%). R_{f} (SiO₂/25% EtOAc in hexanes)=0.34. ¹H NMR (500 MHz, CDCl₃): δ 8.00-7.92 (m, 4H, Ar—H), 7.69 (d, 1H, Ar—H, J=8.3 Hz), 7.59-7.49 (m, 4H, Ar—H), 7.46 (t, 2H, Ar—H, J=7.7 Hz), 7.04 (dt, 1H, Ar—H, J=1.0, 7.4 Hz). ¹³C NMR (125 MHz, CDCl₃): δ 147.9, 134.5, 134.0, 130.6, 130.5, 129.1, 128.9, 128.6, 128.5, 128.1, 127.3, 126.1, 125.2, 123.7, 122.6, 122.2 (t, J=28.6 Hz). ²H NMR (77 MHz, CHCl₃): δ 8.17. HRMS calcd for C₁₈H₁₃DN₃ [M+H]⁺ 273.1245. found 273.1247.

### Example 8

### Preparation of Compound 8: 5-Deutero-1-(4-methoxyphenyl)-4-(N-phthalimidomethyl)-1H-1,2,3-triazole

Compound 8 was prepared by using 4-methoxyphenyl azide and N-phthalimidomethyl ethyne following the general synthetic procedure described above. 4-Methoxyphenyl azide was synthesized from its corresponding boronic acid using the procedures described in Tao et al., Tetrahedron Lett. 2007, 48, 3525-3529. The final product was purified by column chromatography using hexane followed by 20% EtOAc in hexane to give an off-white solid (Yield: 71%; % D: 98%). R_{f} (SiO₂/50% EtOAc in hexanes)=0.40. ¹H NMR (500 MHz, CDCl₃): δ 7.85 (dd, 2H, Ar—H, J=3.1, 5.3 Hz), 7.72 (dd, 2H, Ar—H, J=3.1, 5.3 Hz), 7.57 (d, 2H, Ar—H, J=8.9 Hz), 6.97 (d, 2H, Ar—H, J=8.9 Hz), 5.06 (s, 2H, CH₂), 3.83 (s, 3H, OCH₃). ¹³C NMR (125 MHz, CDCl₃): δ 167.8, 159.9, 143.3, 134.3, 132.2, 130.5, 123.6, 122.4, 121.1 (t, J=28.8 Hz), 114.8, 55.8, 33.2. ²H NMR (77 MHz, CHCl₃): δ 7.97. HRMS calcd for C₁₈H₁₄DN₄O₃ [M+H]⁺ 336.1201. found 336.1204.

### Example 9

### Preparation of Compound 9: 5′-O-(Tert-butyldimethylsilyl)-3′-deoxy-3′-(5-deutero-4-(4-methylphenyl)-1H-1,2,3-triazol-1-yl)thymidine

Compound 9 was prepared by using 0.131 mmol of 3′-azido-3′-deoxy-5′-0-(tert-butyldimethylsilyl) thymidine, 0.262 mmol of 4-methylphenyl ethyne, 10 mol % (relative to the amount of the azide) of anhydrous CuSO₄ in 273 μL of D₂O, 20 mol % (relative to the amount of the azide) of Na ascorbate in 273 μl of D₂O, and 0.65 mL of dry CH₂Cl₂ following the general synthetic procedure described above.

3′-Azido-3′-deoxy-5′-O-(tert-butyldimethylsilyl) thymidine was synthesized using the procedures described in Varizhuk et al., Russ. J. Bioorg. Chem. 2010, 36, 199-206.

The final product was purified by column chromatography using hexane followed by 45% EtOAc in hexane to give a white solid (Yield: 96%; % D: 96%). R_{f} (SiO₂/75% EtOAc in hexanes)=0.48. ¹H NMR (500 MHz, CDCl₃): δ 9.41 (br s, 1H, NH), 7.70 (d, 2H, Ar—H, J=7.9 Hz), 7.49 (s, 1H, Ar—H), 7.22 (d, 2H, Ar—H, J=7.9 Hz), 6.44 (t, 1H, H-1′, J=6.5 Hz), 5.33 (app dt, 1H, H-3′, Jₐₚₚ=4.6, 9.1 Hz), 4.49-4.47 (m, 1H, H-4′), 4.02 (dd, 1H, H-5′, J=2.4, 11.6 Hz), 3.88 (dd, 1H, J=2.4, 11.6 Hz), 3.00 (dt, 1H, H-2′, J=5.9, 13.1 Hz), 2.63 (ddd, 1H, H-2′, J=6.8, 8.5, 14.6 Hz), 2.37 (s, 3H, CH₃), 1.94, (s, 3H, CH₃), 0.93 (s, 9H, tert-Bu), 0.13 and 0.12 (2 s, 6H, SiCH₃). ¹³C NMR (125 MHz, CDCl₃): δ 163.8, 150.5, 148.6, 138.5, 135.5, 129.8, 127.7, 125.9, 118.5 (t, J=28.7 Hz), 111.3, 85.8, 85.0, 62.9, 59.8, 38.8, 26.1, 21.4, 18.6, 12.7, 5.1, 5.2. ²H NMR (77 MHz, CHCl₃): δ 7.85. HRMS calcd for C₂₅H₃₅DN₅O₄Si [M+H]⁺ 499.2594. found 499.2600.

### Example 10

### Preparation of Compound 10: 6-[5-Deutero-4-(4-methylphenyl)-1,2,3-triazol-1-yl]-9-[2,3,5-tri-O-(tert-butyldimethylsilyl)-β-D-ribofuranosyl]purine

Compound 10 was prepared by using 0.198 mmol of 6-azido-9-[2,3,5-tri-0-(tert-butyldimethylsilyl)-β-D-ribofuranosyl]purine, 0.395 mmol of 4-methylphenyl ethyne, 5 mol % (relative to the amount of the azide) of anhydrous CuSO₄ in 0.2 mL of D₂O, 10 mol % (relative to the amount of the azide) of Na ascorbate in 0.2 ml of D₂O, and 0.95 mL of CH₂Cl₂ following the general synthetic procedure described above.

6-Azido-9-[2,3,5-tri-O-(tert-butyldimethylsilyl)-β-D-ribofuranosyl]purine was synthesized using the procedures described in Lakshman et al., J. Org. Chem. 2010, 75, 2461-2473.

The final product was purified by column chromatography using hexane followed by 15% EtOAc in hexane to give a pale-yellow, viscous liquid (Yield: 88%; % D: 95%). R_{f} (SiO₂/20% EtOAc in hexanes)=0.28. ¹H NMR (500 MHz, CDCl₃): δ 8.97 (s, 1H, Ar—H, J=7.8 Hz), 8.65 (s, 1H, Ar—H), 7.91 (d, 1H, Ar—H, J=8.0 Hz), 7.29 (d, 2H, Ar—H, J=8.0 Hz), 6.24 (d, 1H, H-1′, J=5.1 Hz), 4.68 (t, 1H, H-2′, J=4.7 Hz), 4.35 (t, 1H, H-3′, J=3.9 Hz), 4.20 (app q, 1H, H-4′, Jₐₚₚ=3.0 Hz), 4.06 (dd, 1H, H-5′, J=3.5, 11.4 Hz), 3.85 (dd, 1H, H-5′, J=2.4, 11.4 Hz), 2.41 (s, 3H, CH₃), 0.99, 0.96, and 0.81 (3 s, 27H, tert-Bu), 0.19, 0.18, 0.13, 0.12, 0.02, and 0.21 (6 s, 18H, Si—CH₃). ¹³C NMR (125 MHz, CDCl₃): δ 154.5, 152.5, 148.5, 145.2, 145.0, 138.8, 129.8, 127.5, 126.4, 123.5, 119.5 (t, J=26 Hz), 88.9, 86.1, 76.5, 72.3, 62.8, 26.4, 26.1, 25.9, 21.5, 18.8, 18.3, 18.1, 4.1, 4.3, 4.4, 4.6, 4.7, 5.1. ²H NMR (77 MHz, CHCl₃): δ 9.35. HRMS calcd for C₃₇H₆₀DN₇O₄Si₃Na [M+Na]⁺ 775.4048. found 775.4046.

### Example 11

### Preparation of Compound 11: 5-Deutero-1-(3-cyanophenyl)-4-(pyridin-3-yl)-1H-1,2,3-triazole

### Synthesis of 3-Ethynylpyridine

3-Ethynylpyridine was synthesized by using the following procedures:

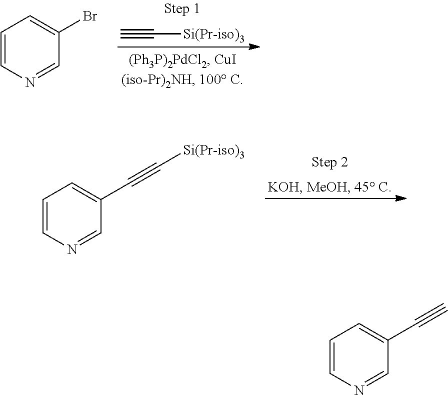

### Step 1. Synthesis of 3-((triisopropylsilyl)ethynyl)pyridine

In a clean, dry three-neck round-bottom flask equipped with a stirring bar, 3-bromopyridine (122 μL, 1.26 mmol) was dissolved in (iso-Pr)₂NH (5 mL). After TIPS-acetylene (336 μL, 1.512 mmol), (Ph₃P)₂PdCl₂ (44.0 mg, 0.063 mmol), and CuI (12.0 mg, 0.063 mmol) were added, the reaction mixture was heated with stirring under a reflux condenser at 100° C. for 1 hour in a nitrogen atmosphere. The reaction mixture was then diluted with EtOAc (20 mL) and filtered through Celite. The filtrate was washed with deionized H₂O (3×20 mL), and brine (15 mL). The organic layer was separated, dried over anhydrous Na₂SO₄, and concentrated under reduced pressure. The crude material was purified by passing it through a short silica gel plug with the initial elution using hexanes followed by 5% EtOAc in hexane to give 294 mg (90% yield) of TIPS-protected ethynylpyridine as a pale-yellow, viscous liquid. R_{f} (SiO₂/10% EtOAc in hexanes)=0.40. ¹H NMR (500 MHz, CDCl₃): δ 8.69 (s, 1H, H-2), 7.51 (d, 1H, H-6, J=4.1 Hz), 7.73 (dt, 1H, H-4, J=1.9, 7.8 Hz), 7.22 (dd, 1H, H-5, J=4.9, 7.8 Hz), 1.13 (s, 21H, Si(iso-Pr)₃). ¹³C NMR (125 MHz, CDCl₃): δ 153.0, 148.8, 139.0, 123.0, 120.9, 103.8, 95.1, 18.7, 11.6. HRMS calcd for C₁₆H₂₆NSi [M+H]⁺ 260.1829. found 260.1716.

### Step 2. Synthesis of 3-ethynylpyridine

In a clean, dry round-bottom flask equipped with a stirring bar, TIPS-protected ethynylpyridine (286 mg, 1.102 mmol) prepared above was dissolved in dry MeOH (15 mL). After powdered KOH (433 mg, 7.72 mmol) was added, the reaction mixture was stirred at 45° C. for 24 hours. To the reaction mixture was added a saturated aqueous NH₄Cl solution, and the mixture was extracted with Et₂O (3×15 mL). The organic layer was separated, washed with brine (10 mL), dried over anhydrous Na₂SO₄, and carefully concentrated by evaporation. The crude material was purified on a short silica gel column using 10% Et₂O in hexane to give 3-ethynylpyridine as a white solid (Yield: 51.0 mg; 45%). R_{f} (SiO₂/20% Et₂O in hexanes)=0.39. ¹H NMR (500 MHz, CDCl₃): δ 8.73 (s, 1H, H-2), 8.57 (d, 1H, H-6, J=4.6 Hz), 7.77 (d, 1H, H-4, J=7.8 Hz), 7.37 (t, 1H, H-5, J=6.4 Hz), 3.22 (s, 1H, ≡CCH).

### Synthesis of Compound 11

Compound 11 was prepared by using 3-cyanophenyl azide and 3-ethynylpyridine following the general synthetic procedure described above. 3-Cyanophenyl azide was synthesized from its corresponding boronic acid using the procedures described in Tao et al., Tetrahedron Lett. 2007, 48, 3525-3529. The final product was purified by column chromatography using 10% EtOAc in hexane followed by EtOAc to give an off-white solid (Yield: 70%; % D: 94%). R_{f} (SiO₂/EtOAc)=0.34. ¹H NMR (500 MHz, CDCl₃): δ 9.17 (br s, 1H, Ar—H), 8.73 (br s, 1H, Ar—H), 8.31 (d, 1H, Ar—H, J=7.9 Hz), 8.15 (t, 1H, Ar—H, J=1.6 Hz), 8.11 (ddd, 1H, Ar—H, J=1.1, 2.2, 8.1 Hz), 7.79 (dt, 1H, Ar—H, J=1.3, 7.7 Hz), 7.74 (t, 1H, Ar—H, J=7.9 Hz), 7.47 (br s, 1H, Ar—H). ¹³C NMR (125 MHz, CD₃OD, 55° C.): δ 150.2, 147.8, 146.8, 139.1, 135.2, 133.7, 132.5, 128.4, 126.1, 125.7, 125.1, 121.2 (t, J=29.6 Hz), 118.7, 115.4. ²H NMR (77 MHz, CHCl₃): δ 8.38. HRMS calcd for C₁₄H₉DN₅ [M+H]⁺ 249.0993. found 249.0860.

### Example 12

### Preparation of Compound 12: 5-Deutero-1-phenyl-D₅-4-(4-methylphenyl)-1H-1,2,3-triazole

### Synthesis of Phenyl azide-D₅

In a clean, dry 2-neck round-bottom flask equipped with a stirring bar, bromobenzene-D₅ (400 mg, 2.468 mmol) was dissolved in 7:3 EtOH/H₂O (4.8 mL). After N,N′-dimethyl ethylenediamine (40 μL, 0.37 mmol), NaN₃ (321 mg, 4.936 mmol), Na ascorbate (24.4 mg, 0.123 mmol), and CuI (47 mg, 0.247 mmol) were added, the mixture was heated with stirring at 100° C. in an argon atmosphere under a reflux condenser for 2 hours. The mixture was then cooled to room temperature and diluted with Et₂O (20 mL) and deionized H₂O (10 mL). The aqueous layer was separated and extracted with Et₂O (3×20 mL). The organic layers were combined, washed with brine (10 mL), dried over anhydrous Na₂SO₄, and concentrated under reduced pressure. The crude material was purified by column chromatography on a short silica gel column using hexane as an eluting solvent to give phenyl azide-D₅ as a yellow liquid (Yield: 261 mg; 85%). R_{f} (SiO₂/hexanes)=0.50. ¹³C NMR (125 MHz, CDCl₃): δ 139.8, 129.2 (t, J=24.2 Hz), 124.3 (t, J=24.2 Hz), 118.6 (t, J=24.2 Hz).

### Synthesis of Compound 12

Compound 12 was prepared by using phenyl azide-D₅ and 4-methylphenyl ethyne following the general synthetic procedure described above. The final product was purified by column chromatography using hexane followed by 20% EtOAc in hexane to give an off-white solid (Yield: 75%; % D: 96%). R_{f} (SiO₂/50% EtOAc in hexanes/)=0.60. ¹H NMR (500 MHz, CDCl₃): δ 7.83 (d, 2H, Ar—H, J=7.8 Hz), 7.29 (d, 2H, Ar—H, J=7.8 Hz), 2.42 (s, 3H, —CH₃). ¹³C NMR (125 MHz, CDCl₃): δ 148.6, 138.5, 137.2, 129.8, 129.5 (t, J=25.0 Hz), 128.4 (t, J=25.0 Hz), 127.6, 126, 120.3 (t, J=24.9 Hz), 117.2 (t, J=29.4 Hz), 21.5. ²H NMR (77 MHz, CHCl₃): δ 8.19, 7.82, 7.58, 7.49. HRMS calcd for C₁₅H₇D₆N₃Na [M+H]⁺ 264.1378. found 264.1299.

### Example 13

### Preparation of Compound 13: 5′-O-(Tert-butyldimethylsilyl)-3′-deoxy-3′-(5-deutero-4-(phenyl-D₅)-1H-1,2,3-triazol-1-yl)thymidine

### Synthesis of Ethynylbenzene-D₅

Ethynylbenzene-D₅ was synthesized by using the following procedures:

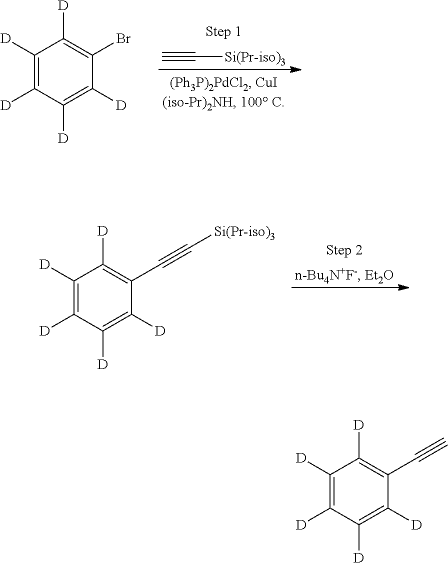

### Step 1. Synthesis of triisopropyl(phenylethynyl)silane-D₅

In a clean, dry 3-neck round-bottom flask equipped with a stirring bar, bromobenzene-D₅ (500 mg, 3.085 mmol) was dissolved in (iso-Pr)₂NH (12.5 mL). After TIPS-acetylene (823 μL, 3.702 mmol), (Ph₃P)₂PdCl₂ (108.3 mg, 0.154 mmol), and Cut (29.4 mg, 0.154 mmol) were added, the reaction mixture was heated with stirring at 100° C. in a nitrogen atmosphere under a reflux condenser for 1.5 hours. The reaction mixture was then diluted with EtOAc (20 mL) and filtered through Celite. The filtrate was washed with deionized H₂O (3×20 mL) and brine (15 mL). The organic layer was separated, dried over anhydrous Na₂SO₄, and concentrated under reduced pressure. The crude material was purified by passing it through a short silica gel plug using hexane as the eluting solvent to give TIPS-protected ethynylbenzene as a colorless viscous liquid (Yield: 500 mg; 61%). R_{f} (hexanes/SiO₂)=0.70. ¹H NMR (500 MHz, CDCl₃): δ 1.16 (s, 21H, Si(iso-Pr)₃). ¹³C NMR (125 MHz, CDCl₃): δ 131.8 (t, J=24.8 Hz), 128.0 (t, J=24.0 Hz), 127.8 (t, J=24.5 Hz), 123.6, 107.3, 90.6, 18.9, 11.6. ²H NMR (77 MHz, CHCl₃): δ 7.51, 7.33, 7.2. HRMS calcd for C₁₇H₂₂D₅Si [M+H]⁺ 264.2190. found 264.2111.

### Step 2. Synthesis of ethynylbenzene-D₅

In a clean, dry 25 mL round-bottom flask equipped with a stirring bar, TIPS-protected ethynylbenzene (700 mg, 2.656 mmol) prepared above was dissolved in dry Et₂O (11 mL) with stirring. After a 1 M solution of n-Bu₄N⁺F⁻ in THF (3.2 mL, 3.2 mmol) was added, the mixture was stirred for 5 minutes. The mixture was then concentrated under a stream of nitrogen gas and loaded onto a short silica gel column. The crude material was purified by chromatograph using hexane as an eluting solvent, followed by careful rotary evaporation of the fractions containing the product using a water bath below 65° C. to give ethynylbenzene-D₅ as a pale-yellow liquid (Yield: 171 mg; 61%). R_{f} (SiO₂/hexanes)=0.42. ¹H NMR (500 MHz, CDCl₃): δ 3.08 (s, 1H, C≡CH). ²H NMR (77 MHz, CHCl₃): δ 7.52, 7.38, 7.35.

### Synthesis of Compound 13

Compound 13 was prepared by using 0.131 mmol of 3′-azido-3′-deoxy-5′-0-(tert-butyldimethylsilyl) thymidine, 0.262 mmol of ethynylbenzene-D₅, 10 mol % (relative to the amount of the azide) of anhydrous CuSO₄ in 0.273 mL of D₂O, 20 mol % (relative to the amount of the azide) of Na ascorbate in 0.273 ml of D₂O, and 0.65 mL of dry CH₂Cl₂ following the general synthetic procedure described above. The azide was prepared by using the method described in Example 9.

The final product was purified by column chromatography using hexane followed by 50% EtOAc in hexane to give a white solid (Yield: 95%; % D: 98%). R_{f} (SiO₂/50% EtOAc in hexanes)=0.21. ¹H NMR (500 MHz, CDCl₃): δ 9.20 (br s, 1H, NH), 7.50 (s, 1H, Ar—H), 6.45 (t, 1H, H-1′, J=6.5 Hz), 5.34 (dt, 1H, H-3′, J=4.6, 8.8 Hz), 4.49-4.51 (m, 1H, H-4′), 4.03 (dd, 1H, H-5′, J=2.1, 11.5 Hz), 3.88 (dd, 1H, H-5′, J=1.8, 11.5 Hz), 3.01 (ddd, 1H, H-2′, J=5.2, 6.3, 14.0 Hz), 2.64 (ddd, 1H, H-2′, J=6.5, 8.1, 14.3 Hz), 1.95 (s, 3H, CH₃), 0.94 (s, 9H, tert-Bu), 0.14 and 0.13 (2 s, 6H, SiCH₃). ¹³C NMR (125 MHz, CDCl₃): δ 163.8, 150.5, 148.4, 135.5, 130.1, 128.6 (t, J=23.8 Hz), 128.1 (t, J=23.0 Hz), 125.6 (t, J=24.1 Hz), 118.7 (t, J=28.8 Hz), 111.4, 85.6, 85.0, 62.8, 59.8, 38.9, 29.9, 26.1, 18.6, 12.8, −5.1, −5.2. ²H NMR (77 MHz, CHCl₃): δ 7.87, 7.45. HRMS calcd for C₂₄H₂₇DN₅O₄SiNa [M+Na]⁺ 512.2571. found 512.2621.

### Example 14

### Preparation of Compound 14: 5-Deutero-1,4-diphenyl-D₁₀-1H-1,2,3-triazole

Compound 14 was prepared by using phenyl azide-D₅ and ethynylbenzene-D₅ following the general synthetic procedure described above. Phenyl azide-D₅ was prepared by using the method described Example 12. Ethynylbenzene-D₅ was prepared by using the method described in Example 11. The final product was purified by column chromatography using hexane followed by 20% EtOAc in hexane to give an off-white solid (Yield: 72%; % D: 94%). R_{f}(SiO₂/50% EtOAc in hexane)=0.60. ¹³C NMR (125 MHz, CDCl₃): δ 148.5, 137.2, 130.3, 129.5 (t, J=24.3 Hz), 128.6 (t, J=23.3 Hz), 128.5 (t, J=23.1 Hz), 128.1 (t, J=21.8 Hz), 125.7 (t, J=24.2 Hz), 120.3 (t, J=25.2 Hz), 117.6 (t, J=26.9 Hz). ²H NMR (77 MHz, CHCl₃): δ 8.23, 7.94, 7.83, 7.58, 7.49. HRMS calcd for C₁₄D₁₁N₃Na [M+H]⁺ 255.1536. found 255.1462.

Assessment of % D in Compound 14.

CH₂Cl₂ (16 μL, 0.25 mmol) was added to the solution of Compound 14 (11.7 mg, 0.05 mmol) in CDCl₃ (0.5 mL). The ¹H NMR of this solution was acquired and the resonances were integrated. The signal at δ 5.30 ppm (CH₂Cl₂) was set to 10 whereby the signal at δ 8.19 ppm (residual triazolyl C5-H) integrated to 0.06. Thus, the % D incorporation in the reaction was determined to be 94%.

### Example 15

### Preparation of Deuterated Compound 1-D and its protio derivative Compound 1-H under different reaction conditions

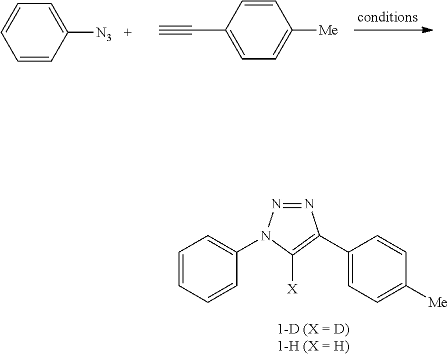

Compound 1 (i.e., 1-D) and its proton derivative (i.e., 1-H) were prepared using general synthetic procedure described above under different reaction conditions. The reaction conditions and results are summarized in Table 1 below.

TABLE 1

| Entry | Solvent, catalytic system, time | Result |
|---|---|---|
| 1 | 1:1 CH₂Cl₂-D₂O, 5 mol % CuSO₄,^{[a]} | 1-D: 60% yield, 80% D |
| | 10 mol % Na ascorbate, 12 hours | |
| 2 | 1:1 CH₂Cl₂-D₂O, 7 mol % CuSO₄,^{[b]} | 1-D: 65% yield, 95% D |
| | 15 mol % Na ascorbate, 48 hours | |
| 3 | 1:1 CH₂Cl₂-D₂O, 10 mol % CuSO₄,^{[b]} | 1-D: 75% yield, 95% D |
| | 20 mol % Na ascorbate, 12 hours | |
| 4 | 1:1 CH₂Cl₂—H₂O, 20 mol % CuCl, | 1-H: about 25% yield^{[c]} |
| | 48 hours | |

^{[a]}CuSO₄ was prepared by heating CuSO₄•5H₂O with a heat gun until the color changed from blue to grey.
^{[b]}CuSO₄ was prepared by heating CuSO₄•5H₂O at 120° C. under vacuum until the color changed from blue to grey.
^{[c]}As estimated by thin layer chromatographic analysis.

As shown in entry 1 in Table 1, use of less stringent conditions (such as simply drying CuSO₄.5H₂O with a heat gun and using a low catalyst load) gave a respectable yield as well as a respectable D incorporation. On the other hand, entries 2 and 3 in Table 1 show that increasing the amount of the catalyst gave much better D incorporation. However, entry 4 in Table 1 shows that CuCl was inferior in catalyzing the reaction between the azide and the alkyne comparing to the CuSO₄/Na ascorbate system.

### Example 16

### Preparation of Deuterated Compound 1-D and its protio derivative Compound 1-H in the presence of 1:1 H₂O/D₂O

Copper-catalyzed reaction of phenyl azide and 4-ethynyl toluene was conducted under the optimized reaction conditions as described in Example 15 above with the exception that 1:1 H₂O/D₂O was used in place of D₂O alone. Under these conditions a significant amount of the protonated 1-H (87%) was formed in comparison to 1-D (13%). A control experiment was conducted side-by-side with only D₂O. The control experiment produced 1-D (97%) with 1-H being a very minor by-product (3%). If one subtracts the 3% formed from the 87% (since the control experiment indicates that 3% will always be produced), then the reaction in 1:1 H₂O/D₂O gave 84% of 1-H and only 16% of 1-D. This clearly shows that the deuteration of a triazole is a more difficult transformation to achieve compared to protonation of the triazole.

Other embodiments are within the scope of the following claims.

1. A method, comprising:
reacting an azide with an alkyne in the presence of deuterated water and a copper-containing catalyst, thereby forming a deuterated 1,2,3-triazole.

2. The method of claim 1, wherein the 1,2,3-triazole contains a deuterium atom at the 5 position.

3. The method of claim 1, wherein the alkyne is ethyne substituted with a substituent comprising C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof.

4. The method of claim 3, wherein the substituent comprises C₁-C₁₀ alkyl, C₁-C₂₀ heterocycloalkyl, aryl, or heteroaryl, which is optionally substituted with D, halo, CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

5. The method of claim 4, wherein the substituent comprises pyridyl, CH₃ substituted with 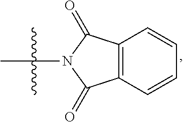 [SMILES: *N1C(=O)c2ccccc2C1=O]
or phenyl optionally substituted with D, F, or CH₃.

6. The method of claim 1, wherein the azide comprises C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group.

7. The method of claim 6, wherein the azide comprises aryl, a nucleoside group, or a deoxynucleoside group, which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

8. The method of claim 7, wherein the azide comprises naphthyl, an adenosine group optionally substituted with C₁-C₁₀ alkylsilyl, C₁-C₁₀ alkyl, or C₁-C₁₀ acyl, a thymidine group optionally substituted with C₁-C₁₀ alkylsilyl, C₁-C₁₀ alkyl, or C₁-C₁₀ acyl, or phenyl optionally substituted with D, CN, C₁-C₁₀ alkyl, or C₁-C₁₀ alkoxy.

9. The method of claim 8, wherein the azide comprises naphthyl, an adenosine group substituted with tert-butyldimethylsilyl, a thymidine group substituted with tert-butyldimethylsilyl, or phenyl optionally substituted with D, CN, tert-butyl, or methoxy.

10. The method of claim 1, wherein the catalyst comprises a copper (I) ion or a copper (II) ion.

11. The method of claim 10, wherein, when the catalyst comprises a copper (II) ion, the reacting step is conducted in the presence of a reducing agent.

12. The method of claim 11, wherein the reducing agent is sodium ascorbate or copper.

13. The method of claim 1, wherein the catalyst is at least about 5 mol % of the azide.

14. The method of claim 1, wherein the reacting step is conducted in the presence of an aprotic solvent.

15. The method of claim 14, wherein the aprotic solvent is immiscible with water.

16. The method of claim 14, wherein the aprotic solvent is methylene chloride, 1,2-dichloroethane, ethyl acetate, chloroform, benzene, an alkyl benzene, or a halo benzene.

17. A method of preparing a deuterated 1,2,3-triazole of formula (I): 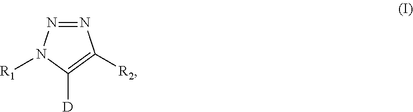
the method comprising:
reacting an azide of formula (II): R₁—N₃ (II) with an alkyne of formula (III):
R₃≡R₂ (III)
in the presence of deuterated water and a copper-containing catalyst, thereby forming the deuterated 1,2,3-triazole of formula (I),
wherein
R₁ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group;
R₂ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof; and
R₃ is H or D.

18. The method of claim 17, wherein R₁ is aryl, a nucleoside group, or a deoxynucleoside group, which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

19. The method of claim 18, wherein R₁ is naphthyl, an adenosine group optionally substituted with C₁-C₁₀ alkylsilyl, C₁-C₁₀ alkyl, or C₁-C₁₀ acyl, a thymidine group optionally substituted with C₁-C₁₀ alkylsilyl, C₁-C₁₀ alkyl, or C₁-C₁₀ acyl, or phenyl optionally substituted with D, CN, C₁-C₁₀ alkyl, or C₁-C₁₀ alkoxy.

20. The method of claim 19, wherein R₁ is naphthyl, an adenosine group substituted with tert-butyldimethylsilyl, a thymidine group substituted with tert-butyldimethylsilyl, or phenyl optionally substituted with D, CN, tert-butyl, or methoxy.

21. The method of claim 17, wherein R₂ is C₁-C₁₀ alkyl, C₁-C₂₀ heterocycloalkyl, aryl, or heteroaryl, which is optionally substituted with D, halo, CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

22. The method of claim 21, wherein R₂ is pyridyl, CH₃ substituted with 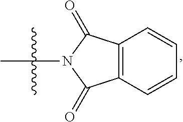 [SMILES: *N1C(=O)c2ccccc2C1=O]
or phenyl optionally substituted with D, F, or CH₃.

23. The method of claim 17, wherein the catalyst comprises a copper (I) ion or a copper (II) ion.

24. The method of claim 23, wherein, when the catalyst comprises a copper (II) ion, the reacting step is conducted in the presence of a reducing agent.

25. The method of claim 24, wherein the reducing agent is sodium ascorbate or copper.

26. The method of claim 17, wherein the catalyst is at least about 5 mol % of the azide.

27. The method of claim 17, wherein the reacting step is conducted in the presence of an aprotic solvent.

28. The method of claim 27, wherein the aprotic solvent is immiscible with water.

29. The method of claim 27, wherein the aprotic solvent is methylene chloride, 1,2-dichloroethane, ethyl acetate, chloroform, benzene, an alkyl benzene, or a halo benzene.

30. A compound of formula (I): 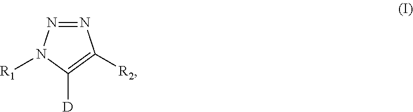
wherein
R₁ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, a nucleoside group, or a deoxynucleoside group, provided that R₁ is not C₁-C₁₀ alkyl substituted with phenyl; and
R₂ is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C₁-C₁₀ alkylsilyl, C₁-C₁₀ alkylstannyl, or a boronic acid group or an ester thereof.

31. The method of claim 30, wherein R₁ is aryl, a nucleoside group, or a deoxynucleoside group, which is optionally substituted with D, halo, CN, OR, SR, N(R)₂, C₁-C₁₀ alkyl, C₁-C₁₀ alkoxy, or C₁-C₁₀ alkylsilyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

32. The method of claim 31, wherein R₁ is naphthyl, an adenosine group optionally substituted with C₁-C₁₀ alkylsilyl, C₁-C₁₀ alkyl, or C₁-C₁₀ acyl, a thymidine group optionally substituted with C₁-C₁₀ alkylsilyl, C₁-C₁₀ alkyl, or C₁-C₁₀ acyl, or phenyl optionally substituted with D, CN, C₁-C₁₀ alkyl, or C₁-C₁₀ alkoxy.

33. The method of claim 32, wherein R₁ is naphthyl, an adenosine group substituted with tert-butyldimethylsilyl, a thymidine group substituted with tert-butyldimethylsilyl, or phenyl optionally substituted with D, CN, tert-butyl, or methoxy.

34. The method of claim 30, wherein R₂ is C₁-C₁₀ alkyl, C₁-C₂₀ heterocycloalkyl, aryl, or heteroaryl, which is optionally substituted with D, halo, CN, OR, SR, N(R)₂, C₁-C₁₀ alkyl or C₁-C₂₀ heterocycloalkyl, R being H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

35. The method of claim 34, wherein R₂ is pyridyl, CH₃ substituted with 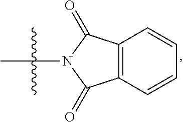 [SMILES: *N1C(=O)c2ccccc2C1=O]
or phenyl optionally substituted with D, F, or CH₃.
